(19) United States
(12) Patent Application Publication
(10) Pub. No.: US 2025/0387546 A1
(43) Pub. Date: Dec. 25, 2025

(54) LUBRICIOUS COATED MEDICAL DEVICES

(71) Applicant: Boston Scientific Scimed, Inc., Maple Grove, MN (US)

(72) Inventors: Justin Theodore Nelson, St. Paul, MN (US); Peter Garrett Zaviska, Mounds View, MN (US); Linda L. Evert, Golden Valley, MN (US); Joseph Thomas Delaney, Jr., Minneapolis, MN (US); Sydney Wallace, Lake Elmo, MN (US); Paul Michael Tomsche, II, Brooklyn Park, MN (US); Michael Frye, Oakdale, MN (US); Riley Derner Duryea, Shakopee, MN (US); Gene Thomas Storbeck, Millis, MA (US); Lauren Sfakis Lydecker, Upton, MA (US); Rick William Dorn, Plymouth, MN (US)

(73) Assignee: Boston Scientific Scimed, Inc., Maple Grove, MN (US)

(21) Appl. No.: 19/243,388

(22) Filed: Jun. 19, 2025

Related U.S. Application Data
(60) Provisional application No. 63/662,315, filed on Jun. 20, 2024.

(51) Int. Cl. A61L 29/14; A61L 29/08

(52) U.S. Cl. CPC A61L 29/14; A61L 29/085

(57) ABSTRACT

In one aspect. the present disclosure pertains to a lubricious coated medical device comprising a substrate and a lubricious coating disposed on the substrate, the lubricious coating comprising a lubricious polyethylene polymer and a non-polar lubricant.

### CROSS-REFERENCE TO RELATED APPLICATIONS

This application claims the benefit of priority under 35 U.S.C. § 119 of U.S.

Provisional Application No. 63/662,315, filed Jun. 20, 2024, the entire disclosure of which is hereby incorporated by reference.

### Technical Field

The disclosure is directed to lubricious coatings. More particularly, the disclosure is directed to lubricious coated medical devices.

### BACKGROUND

A wide variety of intracorporeal medical devices have been developed for medical use, for example, surgical and/or intravascular use. Some of these devices include guidewires, catheters, medical device delivery systems (e.g., for stents, grafts, replacement valves, etc.), and the like. These devices are manufactured by any one of a variety of different manufacturing methods and may be used according to any one of a variety of methods. There is an ongoing need to provide alternative medical devices as well as alternative methods for manufacturing and/or using medical devices. For example, as minimally invasive surgical techniques have improved, it has become increasingly common to insert and retrieve medical devices through catheters and the like having considerable length. Accordingly, it is desirable to minimize friction between the catheters that carry such devices and the devices themselves as well as with tissue with which they may come in contact. In the past, the industry has employed various hydrophobic oils and coatings such as olive oil, silicone, and the like as lubricants. Hydrophilic coatings, particularly hydrogels, also have been employed to impart lubricity to a variety of medical devices.

### SUMMARY

A first example is a lubricious coated medical device is provided. The lubricious coated medical device comprising a substrate and a lubricious coating disposed on the substrate, the lubricious coating comprising a lubricious polyethylene polymer and a non-polar lubricant.

Alternatively or additionally to any of the examples herein, in another example, wherein the lubricious polyethylene polymer has a molecular weight in a range from about 500,000 Daltons to about 11,000,000 Daltons.

Alternatively or additionally to any of the examples herein, in another example, wherein the lubricious polyethylene polymer is an ultra-high molecular weight polyethylene (UHMWPE) having a weight average molecular weight of greater than about 4,000,000 Daltons.

Alternatively or additionally to any of the examples herein, in another example, wherein the lubricious polyethylene polymer is present in a range from about 95 weight percent to about 99.9 weight percent based on a total weight of the lubricious coating.

Alternatively or additionally to any of the examples herein, in another example, wherein the non-polar lubricant is present in a range from about 0.01 weight percent to about 5 weight percent based on the total weight of the lubricious coating.

Alternatively or additionally to any of the examples herein, in another example, wherein the non-polar lubricant has a carbon backbone with 2 to 70 carbon atoms and includes at least one polar head group.

Alternatively or additionally to any of the examples herein, in another example, wherein the non-polar lubricant is selected from a group including: dodecanol, ethoxylated Fatty Alcohol, Ethoxylated Fatty Alcohol, PEG-6 Caprylic/Capric Glycerides, olive oil, avocado oil, coconut oil, ghee, beef tallow, alpha-linolenic acid, paraffin oil, mineral oil, tetrabutylammonium bromide, t-octylphenoxypolyethoxyethanol, hyaluronic acid, poly (4-stryene sulfonic acid), sodium dodecyl sulfonate, oleamide, poly (vinylpyrrolidone), potato starch, ethylene bis stearamide, or any combination thereof.

Alternatively or additionally to any of the examples herein, in another example, wherein the non-polar lubricant is selected from a group including: olive oil, avocado oil, coconut oil, ghee, beef tallow, alpha-linolenic acid, paraffin oil, mineral oil, or any combination thereof.

Alternatively or additionally to any of the examples herein, in another example, wherein the non-polar lubricant is paraffin oil.

Alternatively or additionally to any of the examples herein, in another example, wherein the lubricious coating has a thickness in a range from about 2 micrometers to about 300 micrometers.

Alternatively or additionally to any of the examples herein, in another example, wherein the lubricious coating is formed of a single layer.

Alternatively or additionally to any of the examples herein, in another example, wherein the medical device is per-and polyfluoroalkyl substance (PFAS)-free.

Alternatively or additionally to any of the examples herein, in another example, wherein the lubricious coated medical device has an initial lubricity and durability performance that is substantially equal to or better than an initial lubricity and durability performance of a comparative PFAS coating disposed on the substrate.

Alternatively or additionally to any of the examples herein, in another example, wherein the lubricious coated medical device exhibits continued lubricity and durability performance that is substantially equal to or better than a continued lubricity and durability performance of a comparative PFAS coating disposed on the substrate.

Alternatively or additionally to any of the examples herein, in another example, wherein said medical device is an implantable or insertable medical device.

In another example a lubricious coated medical device is provided. The lubricious coated medical device comprising a substrate material and a lubricious coating disposed on the substrate material, the lubricious coating comprising a lubricious UHMWPE polymer and a non-polar lubricant, wherein the lubricious coating is per- and polyfluoroalkyl substance (PFAS)-free.

Alternatively or additionally to any of the examples herein, in another example, wherein the non-polar lubricant is disposed at least partially within interstitial spaces in the UHMWPE.

In another example, a method of forming a lubricious coated medical device is provided. The method comprising providing a substrate; and i) forming a lubricious coating composition comprising the UHMWPE and a non-polar solvent; applying the lubricious coating composition to the substrate to form a coated medical device; and applying a non-polar lubricant to the coated medical device to form the lubricious coated medical device; or ii) forming a lubricious coating composition comprising an ultra-high molecular weight polyethylene (UHMWPE), the non-polar lubricant, and a non-polar solvent; and applying the lubricious coating composition to the substrate to form the lubricious coated medical device.

Alternatively or additionally to any of the examples herein, in another example, further comprising forming a lubricious coating composition comprising the UHMWPE and a non-polar solvent; applying the lubricious coating composition to the substrate to form a coated medical device; and applying a non-polar lubricant to the coated medical device to form the lubricious coated medical device.

Alternatively or additionally to any of the examples herein, in another example, further comprising forming a lubricious coating composition comprising the UHMWPE, the non-polar lubricant, and a non-polar solvent; and applying the lubricious coating composition to the substrate to form the lubricious coated medical device.

Alternatively or additionally to any of the examples herein, in another example, wherein the applying the lubricious coating composition to the substrate is carried out by a coating method selected from a group including spraying, dipping, brushing, or extruding.

Alternatively or additionally to any of the examples herein, in another example, wherein the applying the non-polar lubricant to the coated medical device is carried out by physically wiping the non-polar lubricant on the coated medical device.

Alternatively or additionally to any of the examples herein, in another example, further comprising irradiating the lubricious coated medical device with about 50 to about 500 kilograys of radiation

The above summary of some example embodiments is not intended to describe each disclosed embodiment or every implementation of the invention.

### BRIEF DESCRIPTION OF THE DRAWINGS

The disclosure may be more completely understood in consideration of the following detailed description of various embodiments in conjunction with the accompanying drawings, in which:

FIG. 1 illustrates the lubricity and durability of various coating compositions; and

FIG. 2 illustrates the lubricity and durability of various coating compositions.

While aspects of the disclosure are amenable to various modifications and alternative forms, specifics thereof have been shown by way of example in the drawings and will be described in detail. It should be understood, however, that the intention is not to limit aspects of the disclosure to the particular embodiments described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

All numeric values are herein assumed to be modified by the term “about”, whether or not explicitly indicated. The term “about” generally refers to a range of numbers that one of skill in the art would consider equivalent to the recited value (i.e., having the same function or result). In many instances, the term “about” may be indicative as including numbers that are rounded to the nearest significant figure.

The recitation of numerical ranges by endpoints includes all numbers within that range (e.g., 1 to 5 includes 1, 1.5, 2, 2.75, 3, 3.80, 4, and 5).

Although some suitable dimensions ranges and/or values pertaining to various components, features and/or specifications are disclosed, one of skill in the art, incited by the present disclosure, would understand desired dimensions, ranges and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

The following detailed description should be read with reference to the drawings in which similar elements in different drawings are numbered the same. The detailed description and the drawings, which are not necessarily to scale, depict illustrative embodiments and are not intended to limit the scope of the invention. The illustrative embodiments depicted are intended only as exemplary. Selected features of any illustrative embodiment may be incorporated into an additional embodiment unless clearly stated to the contrary.

It is generally known to provide substrates, for instance medical devices or parts of such devices, with a coating for the purpose of reducing the friction between medical devices themselves or between a medical device and a tissue when the device is introduced in an aqueous environment, such as the human body, or within another medical device. Such coatings have also been referred to as lubricious or “slippery” coatings. Catheters and other medical devices used for introduction in blood vessels, urethra, body conduits and the like and guide wires used with such devices are examples of articles which may be provided with coatings. Catheters for balloon angioplasty and biopsy are specific examples of such catheters. Other illustrative medical devices may include, but are not limited to, stents, embolic filters, implantable devices, treatment devices, diagnostic devices, guide catheters, sheaths, etc.

One material commonly used to provide a low friction surface is polytetrafluoroethylene (PTFE). However, because of its very low coefficient of friction, it is difficult to wet out the surface of PTFE. Consequently, it can be difficult to adhere other polymers to PTFE, making it difficult to use as a low friction coating. Furthermore, PTFE is very difficult to process. Additionally, use of PTFE along with other per- and polyfluoroalkyl substances (PFAS) may increasingly be subject to various regulatory considerations, environmental concerns, and/or may otherwise be costly. What may be desirable is a lubricious coating that provides similar or improved performance (e.g., lubricity and durability) to traditional coatings such as those that employ PTFE, but is PTFE-free (e.g., does not include any PTFE).

In one example, the present disclosure pertains to lubricious coating compositions for various articles, namely lubricious coated medical devices. The lubricious coating compositions herein, once applied to a substrate and dried, yield lubricious coated medical devices with a lubricious coating. The lubricious coating composition may comprise a mixture of a lubricious polyethylene (e.g., an ultra-high molecular weight polyethylene (UHMWPE))) and a solvent such as non-polar solvent. Optionally, the lubricious coating composition can include a non-polar lubricant. That is, the non-polar lubricant can be applied via a two-step method or a one-shot method, as detailed herein. In any case, the presence of the particular non-polar solvents and the UHMWPE in the relative amounts described herein appear to mitigate or eliminate processing issues that are typically associated with UHMWPE. For instance, the use of unmodified UHMWPE (e.g., UHMWPE alone) and/or the use of UHMWPE without the solvent in the amounts described herein may be suitable for some applications (e.g., forming thick and solid UHMWPE components employed in orthopedic implants via compression molding, etc.). However, such approaches may not be amenable to forming coated medical devices due to processing difficulties associated with forming thin UHMWPE based coatings on substrates. For instance, the lubricious coating compositions herein can be applied in a variety of manners (e.g., dip coating, spray coating, etc.) to form relatively thin lubricious coatings having desirable properties For example, the resultant lubricious coated medical devices exhibit desirable durability and lubricity (e.g., improved durability and lubricity compared to a traditional PTFE coated medical devices), even with the absence of PTFE in the coated medical devices, as detailed herein. Moreover, unlike other coatings, the lubricious coated medical devices herein exhibit a surprising and unexpected increase in lubricity, as detailed herein. While various embodiments herein reference thin UHMWPE based coatings for forming coated medical devices, the approaches herein are also amenable to forming thin UHMWPE based liners suitable for forming lined medical devices, among other types of applications (e.g., heat shrink applications, etc.).

Examples of lubricious polyethylene materials useful in the present disclosure include high density polyethylene (HDPE) and ultra-high molecular weight polyethylene (UHMWPE). In some embodiments, the lubricious polyethylene can have a weight average molecular weight from about 500,000 Daltons to about 11,000,000 Daltons. All individual values and sub-ranges from about 500,000Daltons to about 11,000,000 Daltons are included. For instance, in some embodiments the lubricious polyethylene can have a weight average molecular weight in a range having a lower value of about 500,000 Daltons, about 1,000,000 Daltons, about 2,000,000 Daltons, or about 3,000,000 Daltons and an upper value of about 11,000,000 Daltons, about 10,000,000 Daltons, about 9,000,000 Daltons, about 8,000,000 Daltons, about 7,000,000 Daltons, about 6,000,000 Daltons, about 5,000,000 Daltons, or about 4,000,000 Daltons, among other possibilities. Without wishing to be bound by theory, it is believed that employing a lubricious polyethylene polymer with a higher weight average molecular weight may promote aspects herein. For instance, in some embodiments that lubricious polyethylene polymer can have a weight average molecular weight that is greater than about 4,000,000 Daltons, greater than about 5,000,000 Daltons, greater than about 6,000,000 Daltons, greater than about 7,000,000 Daltons, greater than about 8,000,000 Daltons, greater than about 9,000,000 Daltons, or greater than about 10,000,000 Daltons.

In some embodiments, the lubricious polyethylene polymer can include an UHMWPE. In some embodiments, the lubricious polyethylene polymer can consist essentially of UHMWPE. In such embodiments, the lubricious polyethylene polymer may include a relatively small portion of other components (e.g., less than about 1 weight percent of other components such as another type of polyethylene based on a total weight percent of a polyethylene composition that predominantly includes the UHMWPE). In some embodiments, the lubricious polyethylene polymer can consist of UHMWPE.

Examples of suitable non-polar solvents include decalin, paraffin oil, p-xylene, pentane, hexane, heptane, limonene, dibutyl ketone, isophorone, dodecane, benzene, toluene, acetic acid, chloroform, diethyl ether, ethyl acetate, methylene chloride, dodecane, hexadecane, and pyridine, among others. In some embodiments, the non-polar solvent is selected from a group including decalin, paraffin oil, p-xylene, pentane, hexane, heptane, limonene, dibutyl ketone, isophorone, dodecane, benzene, toluene, acetic acid, chloroform, diethyl ether, ethyl acetate, methylene chloride, dodecane, hexadecane, and pyridine. In some embodiments, the non-polar solvent can be selected from a group including decalin, paraffin oil, p-xylene, along with any combination thereof. In some embodiments, the non-polar solvent can include decalin. In some embodiments, the non-polar solvent can include paraffin oil. In some embodiments, the non-polar solvent can include p-xylene.

In some embodiments, the non-polar solvent can consist essentially of decalin. In some embodiments, the non-polar solvent can consist essentially of paraffin oil. In some embodiments, the non-polar solvent can consist essentially of p-xylene. In some embodiments, the non-polar solvent can consist of decalin. In some embodiments, the non-polar solvent can consist of paraffin oil. In some embodiments, the non-polar solvent can consist of p-xylene.

In some embodiments, the lubricious polyethylene and the non-polar solvent can together form at least 70 weight percent, at least 80 weight percent, at least 90 weight percent, at least 95 weight percent, at least 99 weight percent of a total weight of the lubricious coating composition. For instance, in some embodiments, the lubricious polyethylene and the non-polar solvent can form an entirety (e.g., 100 weight percent) of the lubricious coating composition.

In some embodiments, a mixture of the non-polar solvent and the lubricious polyethylene polymer can be heated to an elevated temperature in a range from about 120 degrees Celsius to about 200 degrees Celsius. All individual ranges and sub-ranges are included. For instance, the mixture of the non-polar solvent and the lubricious polyethylene polymer can be heated to an elevated temperature in a range from about 130 degrees Celsius to about 150 degrees Celsius. In some embodiments the elevated temperature can be about 120 degree Celsius, about 125 degrees Celsius, about 130 degrees Celsius, about 135 degrees Celsius, 140 degrees Celsius, about 145 degrees Celsius, about 150 degrees Celsius, about 155 degrees Celsius, about 160 degrees Celsius, about 165 degrees Celsius, about 170 degrees Celsius, about 175 degrees Celsius, about 180 degrees Celsius, about 185 degrees Celsius, about 190 degrees Celsius, about 195 degrees Celsius, or about 200 degrees Celsius etc.

In some embodiments, the mixture of the non-polar solvent and the lubricious polyethylene polymer can be agitated. For instance, a magnetic stir-bar or other agitation mechanism can be employed to agitate the mixture of the non-polar solvent and the lubricious polyethylene polymer.

Without wishing to be bound by theory, it is believed that heating the mixture of the non-polar solvent and the lubricious polyethylene polymer to an elevated temperature (e.g., an elevated temperature in a range from about 120 degrees Celsius to about 200 degrees Celsius, an elevated temperature in a range from about 120 degrees Celsius to about 160 degrees Celsius, or an elevated temperature in a range from about 130 degrees Celsius to about 150 degrees Celsius) can promote formation of a lubricious coating composition (e.g., promotes dissolving of the lubricious polyethylene polymer in the non-polar solvent), particularly while the mixture is also agitated with an agitation mechanism. For instance, in some embodiments the non-polar solvent can be heated to an elevated temperature and the lubricious polyethylene polymer (e.g., UHMWPE) can subsequently be added to the heated non-polar solvent and the resultant mixture can be agitated and continue to be heated substantially at the elevated temperature until the lubricious polyethylene polymer is dissolved in the non-polar solvent and the lubricious coating composition is formed.

Without wishing to be bound by theory, the presence of the particular non-polar solvents described herein improve processability (e.g., polymer solution rheology) and/or deliverability of the lubricious polyethylene polymer (e.g., UHMWPE) to the substrate. For instance, the resultant lubricious coating composition can be a relatively uniform dispersion of the lubricious polyethylene polymer in the non-polar solvent, and thus can promote aspects herein such as formation of a relatively thin (e.g., about 1 micrometer to about 50 micrometers), yet durable and lubricious coating on a substrate of a medical device. For instance, the lubricious coating composition may be employed to solution cast or otherwise form a thin lubricious coating on the substrate of the substrate of a medical device.

The lubricious coating composition and the resultant lubricious coated medical devices, as detailed herein, are PFAS-free. For instance, unlike some previous approaches and resultant coated medical devices, the lubricious coating composition and the resultant coated medical devices herein are fluorinated ethylene propylene (FEP)-free and PTFE-free. As used herein, being PFAS-free generally refers to including no appreciable or detectable amount of PFAS.

In some embodiments, the lubricious coating compositions herein are applied to the surface of substrate of a medical article as a solution. The lubricious coating compositions herein can include a lubricious polymer, a non-polar solvent, and optionally a non-polar lubricant. For instance, in some embodiments, a non-polar lubricant can subsequently be applied to the surface of the coated surface of the substrate to form a lubricious coated substrate (e.g., a lubricious coated medical device). In some embodiments, the non-polar lubricant can subsequently be applied to the surface of a coated substrate as a liquid or as a solid. However, in some embodiments the non-polar lubricant can be added to the lubricious coating composition and applied to the substrate at the same time (e.g., in one-shot) as the dissolved lubricious polyethylene polymer. For instance, the non-polar lubricant can be added to a lubricious coating composition including a lubricious polyethylene polymer dissolved in a non-polar solvent. The resultant lubricous coating composition including the lubricous polyethylene polymer, the non-polar solvent, and the non-polar lubricant can be applied to the substrate to form a lubricious coated medical device.

In some embodiments, the lubricious polyethylene polymer may be present in a range from about 95 weight percent to about 99.9 weight percent or from about 95 weight percent to about 99 weight percent based on a total weight of the lubricious coating. For instance, in some embodiments, the UHMWPE may be present in a range from about 95 weight percent to about 99.9 weight percent based on a total weight of the lubricious coating. In some embodiments, the non-polar solvent may be present in a range from about 0.01 weight percent to about 5 weight percent, from about 0.1 weight percent to about 5 weight percent, or from about 1 weight percent to about 5 weight percent based on the total weight of the lubricious coating. In some embodiments, a weight ratio of the lubricious polyethylene polymer to the non-polar lubricant (e.g., prior to use and/or testing of a lubricious coated medical device) in the lubricious coating of the lubricious coated medical device may be in a range from about 99:1 to about 19:1. Without wishing to be bound by theory, the non-polar lubricant employed in conjunction with the lubricious polyethylene polymers herein may enhance lubricity to a degree that is comparable to or exceeds lubricity of comparable coated devices (e.g., PTFE coated devices).

The non-polar lubricant can have a carbon backbone with 2 to 70 carbon atoms. All individual values and subranges from 2 to 70 are included. In some embodiments, the non-polar lubricant can have a carbon backbone with 2 to 70 carbon atoms and at least one polar head or polar head group. Stated differently, the carbon backbone can terminate in at least one polar head or polar head group. Without wishing to be bound by theory, it is believed that employing a non-polar lubricant with a carbon backbone with 2 to 70 carbon atoms and at least one polar head can promote aspects herein such as promoting retention of the non-polar lubricant in the coated substrates, thereby enhancing lubricity and durability of the coating on the lubricious coated medical devices herein.

In some embodiments, the non-polar lubricant is selected from a group including: dodecanol, BRIJ L9-LQ-(MV)™, VIRODEX TXR1-LQ-(MV)™, VIRODEX TXR2-LQ-(MH)™, olive oil, avocado oil, coconut oil, ghee, beef tallow, alpha-linolenic acid, paraffin oil, mineral oil, tetrabutylammonium bromide, TRITION X-100™, hyaluronic acid, poly (4-stryene sulfonic acid), sodium dodecyl sulfonate, oleamide, poly (vinylpyrrolidone), potato starch, ethylene bis stearamide, or any combination thereof. In some embodiments, the non-polar lubricant is selected from a group including: olive oil, avocado oil, coconut oil, ghee, beef tallow, dodecanol, alpha-linolenic acid, Macrogol Lauryl Ether 9, Macrogol 6 glycerol caprylocaprate, paraffin oil, xantham gum, tetrabutylammonium bromide, 2-[4-(2,4,4-trimethylpentan-2-yl)phenoxy]ethanol, mineral oil, or any combination thereof. In some embodiments, the non-polar lubricant is selected from a group including olive oil, avocado oil, coconut oil, ghee, beef tallow, dodecanol, alpha-linolenic acid, paraffin oil, mineral oil, or any combination thereof. In some embodiments, the non-polar lubricant is ethylene bis stearamide.

In some embodiments, the non-polar lubricant is selected from a group including olive oil, avocado oil, coconut oil, ghee, beef tallow, paraffin oil, mineral oil, and any combination thereof. In some embodiments, the non-polar lubricant is selected from a group including olive oil, avocado oil, coconut oil, ghee, and beef tallow. In some embodiments, the non-polar lubricant is selected from a group including paraffin oil, mineral oil, or a combination thereof.

In some embodiments, the non-polar lubricant is olive oil. In some embodiments, the non-polar lubricant is avocado oil. In some embodiments the non-polar lubricant is coconut oil. In some embodiments, the non-polar lubricant is ghee. In some embodiments, the non-polar lubricant is beef tallow. In some embodiments, the non-polar lubricant is paraffin oil. In some embodiments, the non-polar lubricant is mineral oil. In some embodiments, the non-polar lubricant is applied to the surface of the coated medial article as a solid (e.g., as a solid wax such as ethylene bis stearamide). In some embodiments, the non-polar lubricant is applied to the surface of the coated medial article as a liquid.

In some embodiments, a hydrophilic polymer may also be included in some lubricious coating compositions (and thus excluded in other compositions). Examples of such polymers, include polyethylene glycol, polypropylene glycol, polyvinylpryrrolidone, and/or hydrophilic urethane polymers, including acrylated urethanes, for example. The polymer may comprise monomer units from one or more monomers having organic acid functional groups. Examples of such monomers include acrylic acid, methacrylic acid, and/or isocrotonic acid.

In some embodiments, a free radical initiator may also be included in some lubricious coating compositions of the present disclosure (and thus excluded in other compositions). The free radical initiator may be, for example, a photoinitiator. Non-limiting examples of free radical photoinitiators that may be employed include benzophenones, ketones, acrylated amine synergists, alpha-amino ketones, acyl phosphine oxides including bis-acyl phosphine oxides, and benzil ketals. More specific examples of photoinitiators suitable for use herein include, but are not limited to, 2-phenyl-1-indanone; IRGACURE 184 from Ciba Specialty Chemicals, BENACURE 184 from Mayzo and SARCURE SR1122 from Sartomer, all of which are 1-hydroxylcyclohexylphenyl ketone (HCPK) initiators; BENACURE BP benzophenone; BENACURE 651 and IRGACURE 651, both of which are benzil dimethyl ketal or 2,2′dimethoxy-2-phenylacetophenone; BENACURE 1732 hydroxy-2-methyl-1-phenyl-1-propanone; IRGACURE 819 bis (2,4,6-trimethylbenzoyl)-phenylphosphineoxide, IRGACURE 907 2-Methyl-1-[4-(methylthio) phenyl]-2-(4-morpholinyl)-1-propanone; IRGACURE 369 morpholinoketone; and so forth and blends thereof. Photoinitiators are also available commercially in a variety of blends. Examples of commercially available blends include, but are not limited to, SARCURE SR1136 is a blend of 4-methylbenzophenone and benzophenone; SARCURE SR1137 is a blend of trimethylbenzophenone and methylbenzophenone; and BENACURE 500, a blend of 1-hydroxylcyclohexylphenyl ketone and benzophenone.

Other optional additives may be used in the lubricious coating compositions of the present disclosure including flow or viscosity modifiers, antioxidants, coupling agents, surfactants, and/or therapeutic agents. Any such additives may be incorporated into the composition at levels of 10 percent or less (e.g., ranging from 10 percent to 5 percent to 2 percent to 1 percent to 0.5 percent or less), based on the dry weight (e.g., excluding solvent) of the composition.

Typically, the lubricious coating compositions for use in the present disclosure may contain from about 0.25 percent to about 10 percent solids, about 0.5 percent to about 10 percent solids, about 1 percent to about 10 percent solids, or about 2 percent to about 7percent solids, or about 3percent to about 6percent solids. The solids of the lubricious coating compositions may include or may be limited to the lubricious polyethylene polymer.

The lubricious coating composition may be applied to the medical device by any method known in the art including, but not limited to, spraying, dipping, rolling, painting (e.g., brush painting, sponge painting, etc.), and so forth. The coating may then be allowed to dry, by evaporation of the solvent at room temperature (e.g., about 20 to 22 degree Celsius). The solvent may be more readily evaporated at an elevated temperature (e.g., a temperature in a range from about 30 degrees Celsius to about 200 degrees Celsius), although room temperature drying is typically acceptable.

A variety of substrate materials may be used in conjunction with the present disclosure including organic and inorganic substrates, typically polymer substrates, metal substrates and glass substrates, among others. Examples of metal substrates include pure metals such as platinum, gold, iridium and titanium, plated metals (e.g., silver plated copper), or metal alloys such as stainless steel including platinum enriched stainless steel (PERSS), Nitinol alloys, and cobalt chromium alloys. An example of a suitable metal substrate is MP35N, a nickel cobalt alloy.

Examples of polymer substrates include the following: (a) olefin homopolymers and copolymers, including homopolymers and copolymers of C2-C8 alkenes, for example, polyethylene and polypropylene, ethylene-vinyl acetate copolymers (EVA), and isobutylene-styrene copolymers, including block copolymers comprising one or more polystyrene blocks and one or more polyisobutylene blocks, for instance, poly (styrene-b-isobutylene-b-styrene) (SIBS), among others, (b) polyamides such as nylons, polyether-polyamide block copolymers such as poly (tetramethylene oxide-b-polyamide-12) block copolymer, available from Elf Atochem as PEBAX, among others, (c) fluoropolymers, including homopolymers and copolymers of C2-C8 alkenes in which one or more hydrogen atoms are substituted with fluorine, for example, polytetrafluoroethylene (PTFE), polyhexafluoropropene (PVDF), polyvinylidene fluoride (PVDF), and poly (vinylidene fluoride-co-hexafluoropropene) (PVDF-HFP), among others, (d) polyurethane copolymers, including copolymers that are polyether based, polyester based, polycarbonate based, aromatic based and aliphatic based, including polyisobutylene based polyurethanes (PIB-PU), (e) silicone homopolymers and copolymers (also referred to as polysiloxanes) such as polydimethylsiloxane, and (f) various thermoplastics such as polyether ether ketone (PEEK).

Examples of substrates include medical article substrates, specific examples of which include medical device substrates, for instance, implantable or insertable medical device substrates. A variety of devices may thus be partially or completely coated with compositions in accordance with the present disclosure, including, for example, catheters (e.g., renal or vascular catheters), balloons, catheter shafts, guide wires, filters (e.g., vena cava filters), stents (including coronary vascular stents, cerebral stents, urethral stents, ureteral stents, biliary stents, tracheal stents, gastrointestinal stents and esophageal stents), stent grafts, cerebral aneurysm filler coils (including Guglilmi detachable coils and metal coils), vascular grafts, myocardial plugs, patches, pacemakers and pacemaker leads, heart valves, vascular valves, tissue engineering scaffolds for cartilage, bone, skin and other in vivo tissue regeneration.

Lubricious coating compositions in accordance with the present disclosure may be cured, for example, by exposing the coating composition to heat or actinic radiation such as UV light for a short period of time and/or allowing drying (solvent evaporation). In some instances, drying can be performed at an elevated temperature. The amount of time needed to dry or cure the surface is dependent on the source of energy, the relative amounts of constituents in the composition, the thickness of the coating desired, and other factors. Generally, the amount of time required for drying or thermal cure can be less than one minute such as about 3 seconds or less at an elevated temperature. Curing around and along the substrate can be accomplished by incrementally or continuously using irradiation from multiple angles using spaced lamps and/or reflectors; rotation of the substrate, light source or light beam; longitudinal movement of the substrate, light source or light beam; or a combination of such techniques.

In some embodiments, a thickness of the lubricious coating on the substrate may be in a range from about 2 micrometers to about 300 micrometers or from about 1 micrometer to about 50 micrometers. In some embodiments, a thickness of the lubricious coating on the substrate may be in the range of from 0.1 micrometers or less to 20 micrometers or more (e.g., from 0.1 to 0.2 to 0.5 to 1 to 2 to 5 to 10 to 20 micrometers), or about 0.1 to about 5 micrometers. The lubricious coating thickness will be affected by the percent solids in the coating and the technique of application, among other factors. Multiple coatings may be applied to achieve a desired coating thickness. However, in some embodiments the lubricious coating can be formed from an individual coating (e.g., an individual application of the lubricious coating composition to the substrate).

In some embodiments, the lubricious coatings herein can be sterilized. In some embodiments the lubricious coatings described herein may comprise a therapeutic agent, for example, selected from antimicrobial agents, antibiotic agents, anti-cancer agents, anti-oxidizing agents (e.g., Vitamin E), agents for treating calcifications, antirestenotic agents and antithrombotic agents, and combinations thereof. The therapeutic agent(s) may be added to the lubricious coating prior to curing or drying or applied onto the coating after it has been dried, cured, and/or sterilized. In some embodiments, therapeutic agent(s) carried in the lubricious polymer coating may remain in the coating or elute out of the coating when the coating is wet, thereby delivering the therapeutic agent(s) to immediately adjacent areas of the body.

It is further contemplated that the present composition may match or increase the lubricity of the lubricious coated medical devices relative to previously known coatings, and may exhibit a further increase in lubricity during use. The increase in lubricity e.g., by at least 10 percent during use is unexpected. That is, traditional coated medical devices tend to lose lubricity during testing/use. Without wishing to be bound by theory it is believed the presence of the non-polar lubricant applied to the lubricious coating including the lubricious polyethylene polymer herein may cause the mechanical testing of the lubricious coated medical device to thereby increase a hardness, increase a density, increase crystallinity, and/or increase the smoothness of an outer surface of the lubricious coated medical device, thereby increasing the lubricity of the coated medical device subsequent to testing/use of the medical device. For instance, the mechanical testing/use may cause the lubricious additive to be disposed at least partially within interstitial spaces in the lubricious polyethylene polymer (e.g., UHMWPE).

In some embodiments, the swelling performance of the implantable or insertable medical devices can be enhanced by irradiation of the implantable or insertable medical devices. For example, lubricious coated medical device i.e., implantable or insertable medical devices that are exposed to various bodily fluids (e.g., bile) and/or various materials (e.g., contrast agents, lubricants, etc.) may exhibit swelling. Such swelling can in turn can reduce the lubricity and durability performance of the implantable or insertable medical devices and/or can result in difficulties deploying, delivering, and/or otherwise using the medical devices. Desirably, it has been found that a degree of swelling of the coated implantable or insertable medical devices can be reduced by irradiation of the implantable or insertable medical devices. The irradiation of the implantable or insertable medical devices with relatively high amounts of radiation e.g., 50 to 500 kilograys can impart crosslinking or an additional degree of crosslinking in the UHMWPE based coatings of the implantable or insertable medical devices which reduces a susceptibility to swelling of the crosslinked coatings of the devices. Such crosslinking appears to alter various physical and/or mechanical properties such as improving the hardness, elastic modulus, etc. of the irradiated devices that in turn reduces that swelling of the coatings of the devices.

For instance, when the irradiated devices (e.g., EX 26-28 which have been irradiated with 100, 200, and 300 kilograys of radiation, respectively) are exposed to various bodily fluids (e.g., bile) and/or various materials (e.g., contrast agents) they desirable exhibit a reduced degree of swelling as compared to implantable or insertable medical devices that have not been irradiated (EX 25-Base UHMWPE), as indicated in Table 2. In some embodiments, irradiation of the implantable or insertable medical devices at the relatively high level of radiation herein (e.g., at least 50 kilograys) can reduce swelling of the irradiated implantable or insertable medical devices by at least 20 percent, by at least 30 percent or by at least 40 percent as compared to comparatively implantable or insertable medical devices that have not been irradiated with such relatively high amounts of radiation. That is, the irradiation of the devices with relatively high amounts of radiation (above amounts of radiation that may be employed in other contexts (e.g., 20 to 40 kilograys for the purpose of sterilization, etc.)) leads to an increased degree of crosslinking and a progressive improvement (reduction) in the swelling performance of the implantable or insertable medical devices. In some embodiments, the lubricious coated medical devices can be exposed to at least 50 kilograys or at least 100 kilograys of radiation to cause or increase a degree of crosslinking of the UHMWPE in the lubricious coated medical devices. In some embodiments, the lubricious coated medical devices can be exposed to 50 to 500 kilograys, 100 to 500 kilograys, or 100 to 300 kilograys of radiation. For instance, the lubricious coated medical devices can be exposed to 100, 200, or 300 kilograys, among other possible values.

The invention is illustrated by the following non-limiting examples.

### Examples 1-24 and Comparative Examples 1-13

The Examples 1-24 and the comparative examples 1-13 were formed as follows. Comparative Example 1 (CE 1): A stainless steel wire available from TEGRA MEDICAL was provided.

Comparative Example 2 (CE 2): A nitinol wire available from FORT WAYNE METALS was provided.

Comparative Example 3 (CE 3): A coated substrate was formed in accordance with the examples 1-12 as described herein, but with the absence of any non-polar lubricant.

### Examples 1-22 (EX 1-22) and Comparative Examples 4-13 (CE 4-13):

A lubricious polyethylene powder (UHMWPE powder with average molecular weight of about 4,200,000 Daltons; tradename GUR2126; available from Celanese) was dissolved in about 1 weight percent decalin at about 130 degrees Celsius under agitation with a magnetic stir bar to form a lubricious coating composition.

A stainless-steel catheter wire (available from TEGRA MEDICAL) was dipped into the lubricious coating composition and allowed to dry to form a coated medical article. This coated medical article corresponds to comparative example 13 (CE 13) in Table 1. The coating thickness for each of the examples was approximately 50.8 micrometers [0.002 inches] or less.

The non-polar lubricants herein were applied in EX 1-22 and CE 4-12 by physically wiping the non-polar lubricants on the coated stainless steel wire to form a lubricious coated medical article. While some of the examples herein apply the non-polar lubricant to the coated substrate to form lubricious coated medical articles, the disclosure is not so limited. Rather, in some examples, the non-polar lubricant can be added to the lubricious coating composition and applied to the substrate at the same time (e.g., in one-shot) as the lubricious polyethylene polymer e.g., as detailed with respect to EX 23-24.

### Examples 23-24 (EX 23-24):

A lubricious coating composition was formed in accordance with CE 13. A non-polar lubricant was added to the lubricious coating composition under agitation with a magnetic stir bar to form a lubricious coating composition including the lubricious polyethylene polymer, the non-polar solvent, and the non-polar lubricant. The lubricious coating composition including the lubricious polyethylene polymer, the non-polar solvent, and the non-polar lubricant was heated to about 135 degrees Celsius. A stainless-steel catheter wire (available from TEGRA MEDICAL) was dipped into the lubricious coating composition including the lubricious polyethylene polymer, the non-polar solvent, and the non-polar lubricant and dried at an elevated temperature (e.g., 30, 90, 150, or 200 degrees Celsius) to form a lubricious coated medical article.

The particular non-polar lubricants employed for each of EX 1-24 and CE 4-12 are provided in Table 1. The coated wires of EX 1-24 and articles of CE 1-13 were tested for lubricity and durability (L&D). To test for L&D, the coated wires of EX 1-24 and the articles of CE 1-13 are secured by a clamping mechanism at a normal force of about 300 grams (g). The force required to pull the coated wires of EX 1-24 and the articles of CE 1-13 through the clamp is measured over a thirty cycle test. Lower frictional force (grams) indicates improved lubricity.

The lubricity and durability testing was conducted under both dry and wet conditions. As used herein, dry conditions refer to contacting the materials under test with a dry silicone pad in the clamping mechanism. As used herein, wet conditions refer to contacting the materials under test with a silicone pad in the clamping mechanism that is immersed in water.

Lubricity and durability performance of the coated wires of EX 1-24 along with EX and the articles of CE 1-13 was determined based on observed frictional forces of the materials undertest after a given quantity of cycles (e.g., after each cycle from 1 to 30 cycles). Unless otherwise indicated, the materials herein were procured from SIGMA-ALDRICH. Xanthan Gum was procured from ICN, Sodium Oleate (powder) was procured from J T BAKER, TRITON X-100™ was procured from MP BIOMEDICALS, sodium 1-dodecansulfonate was procured from BTC, linolenic acid was procured from TCI, and each of Ethoxylated Fatty Alcohol (BRIJ L9-LQ-(MV)™), Ethoxylated Fatty Alcohol (VIRODEX TXR1-LQ-(MV)™), and PEG-6 Caprylic/Capric Glycerides (VIRODEX TXR2-LQ-(MH)™) were procured from CRODA INTERNATIONAL.

TABLE 1
Lubricity and durability performance

| Comparative Examples/ | | | | | | | | | |
|---|---|---|---|---|---|---|---|---|---|
| Examples | | DRY # of Cycles | | | | WET # of Cycles | | | |
| (lubricants indicated | | 1 | 10 | 20 | 30 | 1 | 10 | 20 | 30 |
| where applicable) | | Frictional Force (grams) | | | | | | | |
| CE 1 | Bare wire (O.O22′ SS) | 403 | 374 | 451 | 469 | — | — | — | — |
| CE 2 | Bare wire (nitinol) | — | — | — | — | 553 | 616 | 592 | 598 |
| CE 3 | PTFE coated nitinol | 158 | 129 | 124 | 122 | 190 | 167 | 177 | 185 |
| CE 4 | Poly (acrylic acid) | 574 | 408 | 405 | 398 | 331 | 299 | 302 | 309 |
| CE 5 | Poly (sodium 4-stryene | 468 | 372 | 351 | 341 | 309 | 291 | 302 | 313 |
| | sulfonate) | | | | | | | | |
| CE 6 | Ethoxylated Fatty | 443 | 479 | 498 | 480 | 194 | 218 | 213 | 212 |
| | Alcohol (BRIJ 700) | | | | | | | | |
| CE 7 | Xanthan Gum | 259 | 183 | 164 | 154 | 340 | 312 | 312 | 316 |
| CE 8 | Sodium Oleate | 260 | 243 | 243 | 249 | 310 | 296 | 342 | 358 |
| CE 9 | 2-Hydroxyethyl | 328 | 294 | 300 | 307 | 289 | 305 | 306 | 305 |
| | Cellulose | | | | | | | | |
| CE 10 | PLURONIC F-68 | 399 | 338 | 307 | 286 | 286 | 259 | 265 | 274 |
| CE 11 | PLURONIC F-127 | 476 | 308 | 291 | 280 | 276 | 247 | 246 | 240 |
| CE 12 | SYNPERONIC F 108 | 416 | 290 | 269 | 252 | 228 | 258 | 258 | 259 |
| CE 13 | UHMWPE 0.022″ SS | 248 | 200 | 194 | 198 | 335 | 320 | 300 | 290 |
| EX 1 | Dodecanol | 141 | 47 | 105 | 170 | 102 | 140 | 219 | 232 |
| EX 2 | Ethoxylated Fatty | 119 | 80 | 70 | 68 | 223 | 246 | 236 | 228 |
| | Alcohol | | | | | | | | |
| | (BRIJ L9-LQ-(MV) ™) | | | | | | | | |
| EX 3 | Ethoxylated Fatty | 95 | 69 | 63 | 58 | 193 | 250 | 236 | 226 |
| | Alcohol (VIRODEX | | | | | | | | |
| | TXR1-LQ-(MV) ™) | | | | | | | | |
| EX 4 | PEG-6 Caprylic/Capric | 126 | 90 | 85 | 82 | 250 | 244 | 231 | 227 |
| | Glycerides | | | | | | | | |
| | (VIRODEX TXR2- | | | | | | | | |
| | LQ-(MH) ™) | | | | | | | | |
| EX 5 | Olive Oil | 96 | 70 | 65 | 62 | 76 | 61 | 49 | 43 |
| EX 6 | Avocado Oil | 90 | 70 | 70 | 70 | 135 | 82 | 70 | 68 |
| EX 7 | Coconut Oil | 71 | 67 | 69 | 78 | 50 | 45 | 46 | 45 |
| EX 8 | Ghee | 55 | 47 | 47 | 47 | 28 | 22 | 23 | 23 |
| EX 9 | Beef tallow | 36 | 39 | 39 | 38 | 28 | 46 | 36 | 29 |
| EX 10 | Alpha-linolenic acid | 152 | 45 | 39 | 38 | 79 | 43 | 146 | 177 |
| EX 11 | Paraffin Oil | 149 | 81 | 74 | 70 | 106 | 68 | 59 | 55 |
| EX 12 | Mineral Oil | 128 | 70 | 68 | 66 | 132 | 81 | 75 | 72 |
| EX 13 | Tetrabutylammonium | 220 | 148 | 187 | 132 | 329 | 339 | 360 | 359 |
| | Bromide | | | | | | | | |
| EX 14 | t-Octylphenoxy- | 92 | 76 | 74 | 75 | 192 | 272 | 273 | 297 |
| | polyethoxyethanol | | | | | | | | |
| | (TRITON X-100 ™) | | | | | | | | |
| EX 15 | Hyaluronic acid | 343 | 245 | 223 | 211 | 294 | 286 | 288 | 297 |
| EX 16 | Poly (4-styrene | 326 | 237 | 223 | 211 | 298 | 336 | 343 | 326 |
| | sulfonic acid) | | | | | | | | |
| EX 17 | Sodium dodecyl | 334 | 285 | 266 | 254 | 319 | 288 | 283 | 279 |
| | sulfonate | | | | | | | | |
| EX 18 | Oleamide | 325 | 259 | 227 | 213 | 283 | 273 | 255 | 256 |
| EX 19 | Poly(vinylpyrrolidone) | 309 | 244 | 228 | 219 | 273 | 231 | 173 | 140 |
| EX 20 | Potato Starch | 264 | 199 | 189 | 184 | 337 | 309 | 310 | 315 |
| EX 21 | EBS (dried at 90 | — | — | — | — | 315 | 295 | 294 | 291 |
| | degree Celsius) | | | | | | | | |
| EX 22 | EBS (dried at 150 | — | — | — | — | 305 | 278 | 249 | 237 |
| | degrees Celsius) | | | | | | | | |
| EX 23 | EBS (one-shot | — | — | — | — | 289 | 235 | 234 | 224 |
| | application dried at 90 | | | | | | | | |
| | degrees Celsius) | | | | | | | | |
| EX 24 | EBS (one-shot | — | — | — | — | 287 | 232 | 214 | 208 |
| | application dried at | | | | | | | | |
| | 150 degrees Celsius) | | | | | | | | |

Examples 25-28-Crosslinked swelling and lubricity and durability performance:

The Examples 25-28 were formed as follows.

For each of Examples 25-28, a UHMWPE coated wire samples were created by dissolving 1.0 weigh/volume UHMWPE (GUR 2126 procured from CELANESE) into decahydronaphthalene, 98%, mixture of cis and trans (Fisher Scientific) at 130° C. on a hot plate. A 0.023″ diameter nitinol wire (procured from Fort Wayne Metals) was dipped into the solution and dried at 150° C. for one minute in an oven.

A subset of UHMWPE coated wire samples (EX 26-28) were crosslinked with the application of e-beam irradiation. For examples 26-28, the radiation dosages were 100, 200, and 300 kilograys, respectively. Example 25 was not exposed to a radiation dose.

A solution of bile and contrast was created by dissolving one gram of Ox-bile, dehydrated, purified (procured from MILLIPORE SIGMA) in ten milliliters of phosphate buffered saline (procured from CORNING). Ten milliliters of 240 milligrams iodine/milliliter OMNIPAQUE contrast (procured from GE HEALTHCARE) is mixed into the ox-bile and phosphate buffered saline solution until fully combined.

The outer diameter of each of EX 25-28 was measured on ODAC 33Trio (procured from ZUMBACH) laser measuring device. After the initial measurement, each of coated wires of EX 25-28 was placed into the prepared bile and contrast solution for a period of time (one minute). Each of EX 25-28 was removed from the solution and the outer diameter was measured again. This process of soaking and measuring was repeated at different tests of the coated devices at time intervals of 1, 5, and 10 minutes for each of EX 25-28.

Lubricity and durability testing of EX 25 and EX 28 was performed as described above with respect to EX 1-24 and CE 1-13, with change that the ten cycles were used (rather than thirty cycles) and lubricity and durability measurements were taken for the coated devices of EX 25 and 28 at 0, 1, 5, and 10 minutes of soak time in the prepared bile and contrast solution.

TABLE 2
Crosslinked swelling performance

| | | | | Total Thickness |
|---|---|---|---|---|
| | | | Soak | Change |
| | Radiation | Test | Time | (thousandth |
| Examples | (kilograys) | Number | (minutes) | of an inch) |
| EX 25 | Base UHMWPE | 1 | 0 | 0 |
| | (no radiation) | 2 | 0 | 0 |
| | | 3 | 0 | 0 |
| | | 1 | 1 | 0.13 |
| | | 2 | 1 | 0.17 |
| | | 3 | 1 | 0.13 |
| | | 1 | 5 | 0.27 |
| | | 2 | 5 | 0.22 |
| | | 3 | 5 | 0.29 |
| | | 1 | 10 | 0.24 |
| | | 2 | 10 | 0.29 |
| | | 3 | 10 | 0.35 |
| EX 26 | 100 kilograys of | 1 | 0 | 0 |
| | radiation (kGy) | 2 | 0 | 0 |
| | UHMWPE | 3 | 0 | 0 |
| | | 1 | 1 | 0.07 |
| | | 2 | 1 | 0.16 |
| | | 3 | 1 | 0.19 |
| | | 1 | 5 | 0.07 |
| | | 2 | 5 | 0.11 |
| | | 3 | 5 | 0.13 |
| | | 1 | 10 | 0.14 |
| | | 2 | 10 | 0.19 |
| | | 3 | 10 | 0.2 |
| EX 27 | 200 kGy | 1 | 0 | 0 |
| | UHMWPE | 2 | 0 | 0 |
| | | 3 | 0 | 0 |
| | | 1 | 1 | 0.08 |
| | | 2 | 1 | 0.07 |
| | | 3 | 1 | 0.15 |
| | | 1 | 5 | 0.14 |
| | | 2 | 5 | 0.1 |
| | | 3 | 5 | 0.16 |
| | | 1 | 10 | 0.11 |
| | | 2 | 10 | 0.1 |
| | | 3 | 10 | 0.14 |
| EX 28 | 300 kGy | 1 | 0 | 0 |
| | UHMWPE | 2 | 0 | 0 |
| | | 3 | 0 | 0 |
| | | 1 | 1 | 0.06 |
| | | 2 | 1 | 0.03 |
| | | 3 | 1 | 0.07 |
| | | 1 | 5 | 0.09 |
| | | 2 | 5 | 0.04 |
| | | 3 | 5 | 0.05 |
| | | 1 | 10 | 0.09 |
| | | 2 | 10 | 0.06 |
| | | 3 | 10 | 0.06 |

TABLE 3
Crosslinked lubricity and durability performance

| | | Average |
|---|---|---|
| | | Frictional |
| | | Force (grams) |
| | | of measurements |
| | | taken at 0, 1, 5, |
| | Radiation | and 10 minutes |
| Examples | (kilograys) | of soak time |
| EX 25 | Base UHMWPE - no | 331 |
| | radiation | |
| | Base UHMWPE (with | 93 |
| | Paraffin Oil) | |
| EX 28 | 300 kGy UHMWPE | 277 |
| | 300 kGy UHMWPE | 105 |
| | (with Paraffin Oil) | |

As indicated in Table 1, each of the examples 1-24 (EX 1-24) exhibited satisfactory lubricity and durability performance under at least dry conditions for the entire duration of the lubricity and durability testing (e.g., at each of 1 cycle, 10 cycles, 20 cycles, and 30 cycles).

For instance, each of the examples has an initial lubricity and durability performance that is substantially equal to or better than an initial lubricity and durability performance of a comparative PFAS coating disposed on the substrate, and yet do not employ PTFE. Each of the examples also exhibited continued lubricity and durability performance (e.g., at 10 cycles, 20 cycles, and/or 30 cycles) that is substantially equal to or better than a continued lubricity and durability performance of a comparative PFAS coating disposed on the substrate.

In contrast, each of the comparative examples 1-13 (CE 1-13) employed PTFE and/or exhibited insufficient lubricity performance under least dry conditions during the lubricity and durability testing. For instance, each of the CE 1-13 exhibited higher average frictional force values (where an average friction force value is an average of each of the frication force values for a respective example/comparative example) under dry and/or wet conditions during lubricity and durability testing, as compared to corresponding average friction force values exhibited by each of the EX 1-24 under dry and/or wet conditions during the lubricity and durability testing, as indicated in Table 1. Without wishing to be bound by theory it is believed that at least some of the comparative examples surprisingly exhibited unsatisfactory lubricity and durability performance due at least in part to the presence of the hydrophilic lubricants in the comparative examples.

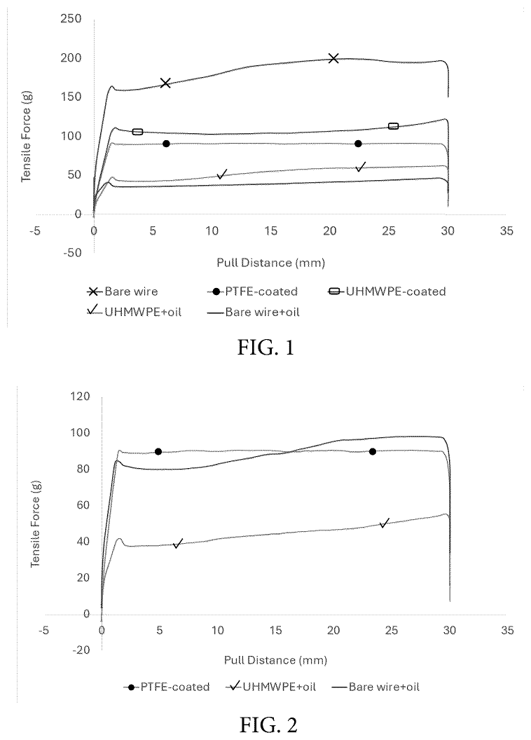

FIG. 1 illustrates a graph of the force required (in grams) to pull an example wire through the clamp for each of the coating compositions under wet conditions over an initial number of cycles (less than 30 cycles). FIG. 2 illustrates a graph of the force required (in grams) to pull the example wire through the clamp for each of the coating compositions under the wet conditions after an increased number of cycles (after 30 cycles). As can be seen in FIG. 1, the example that utilized a lubricious polyethylene polymer (e.g., UHMWPE) with a non-polar solvent in conjunction with the subsequent use a particular non-polar lubricant (paraffin oil) realized comparable lubricity and durability performance to the comparative examples (e.g., PTFE coated medical devices) in a wet environment, despite the absence of PTFE. Similarly, as seen in FIG. 2, the samples that utilized a lubricious polyethylene polymer (e.g., UHMWPE) with a non-polar solvent in conjunction with the subsequent use of a non-polar lubricant herein realize similar lubricity and durability performance compared to the comparative examples (e.g., the PTFE coated medical device of CE 3) in a wet environment, despite the absence of PTFE.

Thus, the lubricious coated medical devices herein can exhibit a lubricity and durability performance that is substantially equal to or better than a lubricity and durability performance of a comparative PFAS coating disposed on the same substrate in at least a dry environment. Depending on a given application having satisfactory lubricity and durability performance in a dry environment but not a wet environment may be suitable. However, in many instances the lubricious coated medical devices exhibited satisfactory lubricity testing in both a dry environment and in a wet environment. Such lubricity and durability performance can be particularly advantageous for the implantable or insertable medical devices herein such as those may encounter fluids during implantation and/or insertion.

In some embodiments, the swelling performance of the implantable or insertable medical devices can be enhanced by irradiation of the implantable or insertable medical devices. For instance, the irradiated device (e.g., EX 28 which was exposed to the highest radiation dosage) still exhibited comparable lubricity and durability performance (e.g., initial lubricity and durability performance) as compared to lubricity and durability performance implantable or insertable medical devices that have not been irradiated (EX 25), as indicated in Table 3. The comparable lubricity and durability performance occurred in both dry and wet (paraffin oil; available as 16512-1L; from SIGMA-ALDRICH) conditions.

Although various embodiments are specifically illustrated and described herein, it will be appreciated that modifications and variations of the present disclosure are covered by the above teachings and are within the purview of the appended claims without departing from the spirit and intended scope of the invention.

What is claimed is:

1. A lubricious coated medical device comprising a substrate and a lubricious coating disposed on the substrate, the lubricious coating comprising a lubricious polyethylene polymer and a non-polar lubricant.

2. The lubricious coated medical device of claim 1, wherein the lubricious polyethylene polymer has a molecular weight in a range from about 500,000 Daltons to about 11,000,000 Daltons.

3. The lubricious coated medical device of claim 1, wherein the lubricious polyethylene polymer is an ultra-high molecular weight polyethylene (UHMWPE) having a weight average molecular weight of greater than about 4,000,000 Daltons.

4. The lubricious coated medical device of claim 1, wherein the lubricious polyethylene polymer is present in a range from about 95 weight percent to about 99.9 weight percent based on a total weight of the lubricious coating.

5. The lubricious coated medical device of claim 1, wherein the non-polar lubricant is present in a range from about 0.01 weight percent to about 5 weight percent based on a total weight of the lubricious coating.

6. The lubricious coated medical device of claim 1, wherein the non-polar lubricant has a carbon backbone with 2 to 70 carbon atoms and includes at least one polar head group.

7. The lubricious coated medical device of claim 1, wherein the non-polar lubricant is selected from a group including: dodecanol, ethoxylated Fatty Alcohol, Ethoxylated Fatty Alcohol, PEG-6 Caprylic/Capric Glycerides, olive oil, avocado oil, coconut oil, ghee, beef tallow, alpha-linolenic acid, paraffin oil, mineral oil, tetrabutylammonium bromide, t-octylphenoxypolyethoxyethanol, hyaluronic acid, poly (4-stryene sulfonic acid), sodium dodecyl sulfonate, oleamide, poly(vinylpyrrolidone), potato starch, ethylene bis stearamide, or any combination thereof.

8. The lubricious coated medical device of claim 1, wherein the non-polar lubricant is selected from a group including: olive oil, avocado oil, coconut oil, ghee, beef tallow, alpha-linolenic acid, paraffin oil, mineral oil, or any combination thereof.

9. The lubricious coated medical device of claim 1, wherein the non-polar lubricant is paraffin oil.

10. The lubricious coated medical device of claim 1, wherein the lubricious coating has a thickness in a range from about 2 micrometers to about 300 micrometers.

11. The lubricious coated medical device of claim 1, wherein the lubricious coating is formed of a single layer.

12. The lubricious coated medical device of claim 1, wherein the medical device is per-and polyfluoroalkyl substance (PFAS)-free.

13. The lubricious coated medical device of claim 1, wherein the lubricious coated medical device has an initial lubricity and durability performance that is substantially equal to or better than an initial lubricity and durability performance of a comparative PFAS coating disposed on the substrate.

14. The lubricious coated medical device of claim 1, wherein the lubricious coated medical device exhibits continued lubricity and durability performance that is substantially equal to or better than a continued lubricity and durability performance of a comparative PFAS coating disposed on the substrate.

15. The lubricious coated medical device of claim 1, wherein said medical device is an implantable or insertable medical device.

16. A lubricious coated medical device comprising a substrate material and a lubricious coating disposed on the substrate material, the lubricious coating comprising an ultra-high molecular weight polyethylene (UHMWPE) and a non-polar lubricant, wherein the lubricious coating is per-and polyfluoroalkyl substance (PFAS)-free.

17. A method of forming a lubricious coated medical device, the method comprising:
providing a substrate; and
i) forming a lubricious coating composition comprising the UHMWPE and a non-polar solvent;
applying the lubricious coating composition to the substrate to form a coated medical device; and
applying a non-polar lubricant to the coated medical device to form the lubricious coated medical device; or
ii) forming a lubricious coating composition comprising an ultra-high molecular weight polyethylene (UHMWPE), the non-polar lubricant, and a non-polar solvent; and
applying the lubricious coating composition to the substrate to form the lubricious coated medical device.

18. The method of claim 17, further comprising:
forming a lubricious coating composition comprising the UHMWPE and a non-polar solvent;
applying the lubricious coating composition to the substrate to form a coated medical device; and
applying a non-polar lubricant to the coated medical device to form the lubricious coated medical device.

19. The method of claim 17, further comprising:
forming a lubricious coating composition comprising the UHMWPE, the non-polar lubricant, and a non-polar solvent; and
applying the lubricious coating composition to the substrate to form the lubricious coated medical device.

20. The method of claim 17, further comprising irradiating the lubricious coated medical device with about 50 to about 500 kilograys of radiation.
